(19) United States
(12) Patent Application Publication
(10) Pub. No.: US 2026/0168042 A1
(43) Pub. Date: Jun. 18, 2026

(54) COMPOSITIONS AND METHODS FOR THE DETECTION AND ANALYSIS OF HERPES SIMPLEX VIRUS 1 (HSV-1), HERPES SIMPLEX VIRUS 2 (HSV-2) AND VARICELLA ZOSTER VIRUS (VZV)

(71) Applicant: Abbott Molecular, Inc., Des Plaines, IL (US)

(72) Inventors: Jeffrey D. Wuitschick, Wauwatosa, WI (US); Rupinder Kular, Chicago, IL (US); Tomasz Krupinski, Des Plaines, IL (US); Heather C. Alexander, Chicago, IL (US); Qingbei Zhang, Park Ridge, IL (US)

(21) Appl. No.: 19/125,936

(22) PCT Filed: Nov. 3, 2023
(86) PCT No.: PCT/US2023/036756

§ 371 Date: Apr. 30, 2025

Related U.S. Application Data
(60) Provisional application No. 63/422,599, filed on Nov. 4, 2022.
(60) Provisional application No. 63/430,958, filed on Dec. 7, 2022.

(51) Int. Cl. C12Q 1/70; B01L 3/00; B01L 7/00; C12N 15/10; C12Q 1/6806; C12Q 1/686

(52) U.S. Cl. CPC C12Q 1/705; B01L 3/502761; B01L 7/52; C12N 15/1013; C12Q 1/6806; C12Q 1/686; B01L 2200/16; B01L 2300/0829; B01L 2300/18; C12Q 2600/16; C12Q 2600/166

(57) ABSTRACT

Provided herein are compositions and methods useful for the detection of Herpes Simplex Virus 1 (HSV-1), Herpes Simplex Virus 2 (HSV-2) and Varicella Zoster Virus (VZV). In particular, provided herein are compositions, methods, systems, kits, reagents, and reaction mixtures involving such for nucleic acid amplification and detection procedures that detect HSV-1, HSV-2 and VZV in samples.

### SEQUENCE LISTING

The text of the computer readable sequence listing filed herewith, titled “40733-601_SEQUENCE_LISTING”, created Nov. 3, 2023, having a file size of 19,615 bytes, is hereby incorporated by reference in its entirety.

### FIELD OF THE INVENTION

Provided herein are compositions and methods useful for the detection of Herpes Simplex Virus 1 (HSV-1), Herpes Simplex Virus 2 (HSV-2) and Varicella Zoster Virus (VZV). In particular, provided herein are compositions, methods, systems, kits, reagents, and reaction mixtures involving such for nucleic acid amplification and detection procedures that detect HSV-1, HSV-2 and VZV in samples.

### BACKGROUND OF THE INVENTION

Human herpes viruses are divided into alpha, beta and gamma subtypes. Alpha Herpesviridae includes Herpes Simplex Virus 1 (HSV-1), Herpes Simplex Virus 2 (HSV-2) and Varicella Zoster Virus (VZV). HSV-1 and HSV-2 are large double-stranded DNA viruses and share a similar genome with approximately 80% homology of their protein-coding regions. HSV-1, HSV-2 and VZV cause a variety of illnesses depending on the anatomical site where the infection begins, the immune status of the host, and whether the infection is primary or recurrent. Upon primary infection, HSV enters through the skin or mucosa, infects epithelial cells and begins replicating. Following infection, the virus may become latent in local sensory ganglions periodically reactivating to cause symptomatic lesions. HSV-1 is the causative agent of “cold sores”, i.e., vesicular lesions of the oral mucosa, and can also be transmitted to genitals through oral-genital contact and cause clinical disease in the genitalia. HSV-2 is transmitted sexually with lesions that are primarily localized to the anogenital anatomical areas. Primary VZV infection most often occurs during childhood and causes varicella (chickenpox), and latent infection in sensory neurons in the dorsal root ganglia (DRG) along the neuroaxis. When virus-specific cellular immunity wanes during ageing or as a result of immunosuppression, a reactivation of the latent infection with replication of VZV in 1 or more DRGs causes Herpes zoster (HZ, Shingles).

Accordingly, a rapid and precise diagnostic test able to detect, identify and distinguish HSV-1, HSV-2 and VZV from one another is needed.

### SUMMARY OF THE INVENTION

Provided herein are compositions and methods useful for the detection of Herpes Simplex Virus 1 (HSV-1), Herpes Simplex Virus 2 (HSV-2) and Varicella Zoster Virus (VZV).

In particular, provided herein are compositions, methods, systems, kits, reagents, and reaction mixtures involving such for nucleic acid amplification and detection procedures that detect HSV-1, HSV-2 and VZV in samples.

Such compositions and methods comprise primers, probes, primer sets, primer and probe sets, and methods for detecting HSV-1, HSV-2 and VZV complex in different human samples including, for example, cutaneous and mucocutaneous lesion swab samples, cerebrospinal fluid, plasma and serum.

In some embodiments, two or more of the polynucleotide reagents provided herein as SEQ ID NOs: 1-21 are combined in a composition (e.g., reagent set, kit, reaction mixture, system, etc.). In some embodiments, one or more or all of the nucleic acid reagents comprise a detectable moiety (e.g., a synthetic label). In some embodiments, the compositions comprise one or more primer pairs of SEQ ID NOs: 1 and 2, 4 and 5, 7 and 8, 10 and 11, 13 and 14, 16 and 17, and 19 and 20. In some embodiments, the compositions comprise one or more probes (e.g., labeled probes) of SEQ ID NOs: 3, 6, 9, 12, 15, 18 and 21. In some embodiments, the compositions comprise primer and probe sets: SEQ ID NOs: 1-2 and 3, 4-5 and 6, or 7-8 and 9, 10-11 and 12, 13-14 and 15, 16-17 and 18, and/or 19-20 and 21. In some embodiments, the compositions comprise internal control reagents, such as SEQ ID NOs: 19-21. In some embodiments, the compositions comprise a multi-probe system comprising SEQ ID NOs: 3, 6, 9, 12, 15, 18 and 21.

In some embodiments, the compositions and methods of the present invention employ reagent sets comprising a polynucleotide component having primers, probes, primer sets, and/or probe sets. In some embodiments, the polynucleotide component of the composition consists of the primer, probe, primer set, or probe set combinations described above. As reaction mixtures, the compositions may consist of such polynucleotides, as well as any polynucleotides included in a sample (i.e., the only non-sample nucleic acid molecules are the polynucleotides represented by SEQ ID NOs: 1-21, individually or in combinations (e.g., the combinations described above)).

The primer sets herein provided comprise two primers, and are useful for the amplification of target sequences, e.g., in polymerase chain reaction (PCR) amplification. In some embodiments, the compositions comprise at least two primers and one or more (e.g., two or more) probes that detect amplicons generated by the primers.

Also provided herein are methods for detecting HSV-1, HSV-2 and VZV in a sample. In some embodiments, the methods comprise (a) forming a reaction mixture comprising nucleic acid amplification reagents, at least one polynucleotide primer or probe described herein, and a test sample potentially containing at least one target sequence; and (b) subjecting the mixture to amplification conditions to generate at least one copy of a nucleic acid sequence complementary to the target sequence. In some embodiments the method further comprises detecting generated amplicons. In some embodiments, the detecting comprises (c) hybridizing a probe to the nucleic acid sequence complementary to the target sequence so as to form a hybrid comprising the probe and the nucleic acid sequence complementary to the target sequence; and (d) detecting, directly or indirectly, the hybrid as an indication of the presence of HSV-1, HSV-2 and VZV in the test sample.

Further, when the amplification is PCR, or a similar thermocycling amplification process, step (b) can be repeated multiple times to increase the number of target sequence copies.

According to another embodiment, both HSV-1, HSV-2 and VZV and one or more additional infectious agents (e.g., HIV) or other nucleic acid molecules (e.g., human sequences) are detected. Accordingly, in some embodiments, compositions comprise reagents for detecting such other agents or nucleic acid molecules.

In some embodiments, the compositions and methods further employ control reagents and/or kit components (e.g., positive controls and/or negative controls). In some embodiments, the control reagents include a synthetic target nucleic acid. In some embodiments, the control reagents include reagents for detecting an HSV-1, HSV-2 and VZV, human, or other sequence expected to be present in a sample. In some embodiments, a control target nucleic acid, whether synthetic or endogenous in a sample, is selected such that amplification primers that amplify the HSV-1, HSV-2 and VZV target nucleic acid also amplify the control target nucleic acid. In some such embodiments, a probe that detects the HSV-1, HSV-2 and VZV target nucleic acid, or an amplicon generated therefrom does not detect the control target or an amplicon generated therefrom. In some embodiments, a control probe is provided that detects the control target nucleic acid or an amplicon generated therefrom, but does not detect the HSV-1, HSV-2 and VZV target nucleic acid, or an amplicon generated therefrom. In some embodiments, internal standards and calibrants are provided for quantitation.

In some embodiments, kits, in addition to the reagents discussed above, include one or more suitable containers, instructions for use, software (e.g., data analysis software), instructions and the like. In some embodiments, kits include reagents for labeling polynucleotides. In some embodiments, one or more components in the kit is in lyophilized form. In other embodiments, one, or more, or all reagents of the present invention are in liquid form.

Embodiments of the present disclosure provide compositions, kits, systems, and methods for identifying HSV-1, HSV-2 and VZV in complex biological samples such as a skin sample, a mucosal sample, a surface swab sample, a cerebrospinal fluid sample, a plasma sample, an oral sample, a lesion sample, a biopsy sample, an anogenital sample, and/or a blood sample. In some embodiments, the compositions and methods provide one or more single probes for real time detection methods that are able to specifically and accurately isolate and identify HSV-1, HSV-2 and VZV.

For example, in some embodiments, the present disclosure provides a composition, comprising: at least one (e.g., one, two, or three) primer pair(s) selected from SEQ ID NOs: 1 and 2, 4 and 5, 7 and 8, 10 and 11, 13 and 14, 16 and 17, and 19 and 20. In some embodiments, the composition further comprises at least one probe selected from SEQ ID NOs: 3, 6, 9, 12, 15, 18 and 21.

Additional embodiments provide a composition, comprising each of the nucleic acids of SEQ ID NOs: 1-21.

Embodiments of the disclosure provide a kit, comprising: a) any of the aforementioned compositions, and b) at least one reagent for performing a nucleic acid amplification reaction (e.g., a nucleic acid polymerase; a plurality of dNTPS, and/or a buffer)

In other embodiments, the disclosure provides a reaction mixture, comprising: any of the aforementioned compositions or nucleic acids hybridized to a HSV-1, HSV-2 and VZV nucleic acid. In some embodiments, the HSV-1, HSV-2 and VZV target nucleic acid is one or more of HSV-1 US-6, HSV-1 UL-1, HSV-2 UL-1, HSV-2 UL-18, VZV ORF10, and/or VZV ORF21.

In further embodiments, the present disclosure provides a method of identifying an HSV-1, HSV-2 and VZV nucleic acid in a biological sample, comprising: a) contacting a biological sample from a subject with any of the aforementioned nucleic acid primers or probes, and b) directly or indirectly detecting the binding of the nucleic acid primers or probes to the HSV-1, HSV-2 and VZV nucleic acid. In some embodiments, the method further comprises the step of c) determining the presence of HSV-1, HSV-2 and VZV in the sample when the binding is detected. In some embodiments, the detecting is via real time PCR detecting, also referred to as quantitative PCR (qPCR).

Yet other embodiments provide a method of detecting an HSV-1, HSV-2 and VZV nucleic acid in a biological sample, comprising: a) extracting DNA from the sample; b) contacting the DNA with one or more primer pairs and one or more nucleic acid probes; c) performing an amplification assay to amplify one or more HSV-1, HSV-2 and VZV nucleic acid targets; and d) identifying the presence of the targets in the sample.

Further embodiments provide a method of detecting an HSV-1, HSV-2 and/or VZV nucleic acid in a biological sample, comprising: a) extracting DNA from the sample; b) contacting the DNA with one or more primer pairs selected from SEQ ID NOs: 1 and 2, 4 and 5, 7 and 8, 10 and 11, 13 and 14, 16 and 17, and 19 and 20; and one or more nucleic acid probes selected from SEQ ID NOs: 3, 6, 9, 12, 15, 18 and 21; c) performing an amplification assay to amplify one or more HSV-1, HSV-2 and VZV nucleic acid targets; and d) identifying the presence of the targets in said sample.

Further embodiments provide a method of detecting an HSV-1, HSV-2 and/or VZV nucleic acid in a biological sample, comprising: a) extracting DNA from the sample; b) contacting the DNA with one or more primer pairs selected from SEQ ID NOs: 1 and 2, 4 and 5, 7 and 8, 10 and 11, 13 and 14, 16 and 17, and 19 and 20; and one or more nucleic acid probes selected from SEQ ID NOs: 3, 6, 9, 12, 15, 18 and 21; c) performing real-time polymerase chain assay to amplify one or more HSV-1, HSV-2 and VZV nucleic acid targets; and d) identifying the presence of the targets in said sample.

In some embodiments, the methods, compositions, kits, reaction mixtures, and systems of the present invention provide an automated multiplex assay for qualitative detection and differentiation of HSV-1, HSV-2 and VZV from cutaneous and mucocutaneous lesion specimens. In some embodiments, the present invention provides an aid in the diagnosis and care of persons with HSV-1, HSV-2 and VZV infections.

Additional embodiments are described herein.

### DETAILED DESCRIPTION

Provided herein are compositions and methods useful for the detection of Herpes Simplex Virus 1 (HSV-1), Herpes Simplex Virus 2 (HSV-2) and Varicella Zoster Virus (VZV). In particular, provided herein are compositions, methods, systems, kits, reagents, and reaction mixtures involving such for nucleic acid amplification and detection procedures that detect HSV-1, HSV-2 and VZV in samples.

In some embodiments, the present invention provides two sets of primers and probes for detection of HSV-1, two sets of primers and probes for detection of HSV-2 and two sets of primers and probes for detection of VZV. In addition to the primers and probes, the present invention comprises a diagnostic method and assay format that uses the above-mentioned primers and probes to achieve specific and sensitive detection of HSV-1, HSV-2 and VZV using real-time PCR technology on, for example, the Alinity m® system. In some embodiments, the present invention further provides an internal control that serves as validity control for sample processing and amplification efficiency.

Multiple strains of HSV-1, HSV-2 and VZV are prevalent among human populations, and the genomic sequences of different strains may exhibit genetic variability. Such natural polymorphisms within primer probe binding sites can result in inefficient hybridization and lead to lack of detection for a nucleic acid single-plex test method based on the PCR technology. To ensure assay robustness, HSV 1 & 2 and VZV assays of the present invention are configured to target two conserved sequences within the respective viral genome i.e., dual-target testing for each target virus. In some embodiments, for HSV-1 the gene targets are Glycoprotein D (US6)+Unique Long Region (UL1), for HSV-2 the gene targets are UL1+UL18, and for VZV the gene targets are Open Reading Frame (ORF) 10+ORF 21.

In some embodiments, provided herein are polynucleotides that specifically hybridize with a nucleic acid sequence, or complement thereof, of HSV-1, HSV-2 and VZV. These polynucleotides find use to amplify HSV-1, HSV-2 and VZV if present in a sample, and to specifically detect the presence of HSV-1, HSV-2 and VZV. Exemplary polynucleotides are described, for example, by SEQ ID NOs: 1-18.

In some embodiments, assays described herein utilize multiple (e.g., two) different HSV-1, HSV-2 and VZV -specific primer/probe sets. For example, in some embodiments, a first set is designed to detect the one gene and second set, is designed to detect another gene. Experiments described herein demonstrate that the dual target strategy of the present invention results in the detection of HSV-1, HSV-2 and VZV genomic DNA with high reliability.

Embodiments of the technology described herein provide high throughput, automated HSV-1, HSV-2 and VZV detection with high sensitivity and specificity.

The term “specifically hybridize” as used herein refers to the ability of a nucleic acid to bind detectably and specifically to a second nucleic acid. Polynucleotides specifically hybridize with target nucleic acid strands under hybridization and wash conditions that minimize appreciable amounts of detectable binding to non-specific nucleic acids. Stringent conditions that can be used to achieve specific hybridization are known in the art.

A “target sequence” or “target nucleic acid sequence” as used herein means a nucleic acid sequence of HSV-1, HSV-2 and VZV or other sequence to be detected (e.g., HIV), or complement thereof, that is amplified, detected, or both amplified and detected using one or more of the polynucleotides herein provided. Additionally, while the term target sequence sometimes refers to a double stranded nucleic acid sequence, those skilled in the art will recognize that the target sequence can also be single stranded. In cases where the target is double stranded, polynucleotide primer sequences preferably amplify both strands of the target sequence. A target sequence may be selected that is more or less specific for a particular organism. For example, the target sequence may be specific to an entire genus, to more than one genus, to a species or subspecies, serogroup, auxotype, serotype, strain, isolate or other subset of organisms.

The term “test sample” as used herein, means a sample taken from an organism, biological fluid, environmental sample, or other sample that is suspected of containing or potentially contains an HSV-1, HSV-2 and VZV target sequence. The test sample can be taken from any biological source, such as for example, tissue, blood, saliva, sputa, mucus, bronchial sweat, urine, urethral swabs, cervical swabs, urogenital or anal swabs, lesion swabs, conjunctival swabs, ocular lens fluid, cerebral spinal fluid, milk, ascites fluid, synovial fluid, peritoneal fluid, amniotic fluid, fermentation broths, cell cultures, tissue biopsies, chemical reaction mixtures and the like. The test sample can be used (i) directly as obtained from the source or (ii) following a pre-treatment to modify the character of the sample. Thus, the test sample can be pre-treated prior to use by, for example, preparing plasma or serum from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

The term “label” as used herein means a molecule or moiety having a property or characteristic which is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate”. A conjugate is typically a specific binding member that has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, e.g., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

A polynucleotide is a nucleic acid polymer of ribonucleic acid (RNA), deoxyribonucleic acid (DNA), modified RNA or DNA, or RNA or DNA mimetics (such as, without limitation PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” For ease of preparation and familiarity to the skilled artisan, polynucleotides are preferably modified or unmodified polymers of deoxyribonucleic acid or ribonucleic acid.

Polynucleotide analogues that are useful include polymers with modified backbones or non-natural inter-nucleoside linkages. Modified backbones include those retaining a phosphorus atom in the backbone, such as phosphorothioates, chiral phosphorothioates, phosphorodithioates, phosphotriesters, aminoalkylphosphotriesters, methyl and other alkyl phosphonates, as well as those no longer having a phosphorus atom, such as backbones formed by short chain alkyl or cycloalkyl internucleoside linkages, mixed heteroatom and alkyl or cycloalkyl internucleoside linkages, or one or more short chain heteroatomic or heterocyclic internucleoside linkages. An example of such a non-phosphorus containing backbone is a morpholino linkage (see, for example, U.S. Pat. Nos. 5,185,444, 5,034,506, and 5,142,047 all of which are herein incorporated by reference). Modified nucleic acid polymers (analogues) may contain one or more modified sugar moieties. For example, sugar moieties may be modified by substitution at the 2′ position with a 2-methoxyethoxy (2-MOE) group (see, for example, Martin et al., (1995) Helv. Chim. Acta, 78:486-504).

Embodiments also contemplate analogues that are RNA or DNA mimetics, in which both the sugar and the inter-nucleoside linkage of the nucleotide units are replaced with novel groups. In these mimetics the base units are maintained for hybridization with the target sequence. An example of such a mimetic, which has been shown to have excellent hybridization properties, is a peptide nucleic acid (PNA) (Nielsen et al., (1991) Science, 254:1497-1500; International Patent Application WO 92/20702, herein incorporated by reference). In PNA compounds, the sugar-backbone of an oligonucleotide is replaced with an amide containing backbone, for example an aminoethylglycine backbone. The nucleobases are retained and are bound directly or indirectly to the aza-nitrogen atoms of the amide portion of the backbone.

Contemplated polynucleotides may further include derivatives wherein the nucleic acid molecule has been covalently modified by substitution, chemical, enzymatic, or other appropriate means with a moiety other than a naturally occurring nucleotide, for example with a moiety that functions as a label, as described herein.

The present invention further encompasses homologues of the polynucleotides having nucleic acid sequences set forth in SEQ ID NOs: 1-21. Homologues are nucleic acids having at least one alteration in the primary sequence set forth in any one of SEQ ID NOs: 1-21 that does not destroy the ability of the polynucleotide to specifically hybridize with a target sequence, as described above. Accordingly, a primary sequence can be altered, for example, by the insertion, addition, deletion or substitution of one or more of the nucleotides of, for example, SEQ ID NOs: 1-21. Thus, homologues that are fragments of a sequence disclosed in SEQ ID NOs: 1-21 may have a consecutive sequence of at least about 7, 10, 13, 14, 15, 16, 17, 18, 19 20, 21, 22, 23 or more nucleotides of the nucleic acid sequences of SEQ ID NO: 1-21, and will retain the ability to specifically hybridize with a target sequence, as described above. Ordinarily, the homologues will have a nucleic acid sequence having at least about 50%, 60%, 70%, 80%, 85%, 90% or 95% nucleic acid sequence identity with a nucleic acid sequence set forth in SEQ ID NOs: 1-21. Identity with respect to such sequences is defined herein as the percentage of nucleotides in the candidate sequence that are identical with the known polynucleotides after aligning the sequences and introducing gaps, if necessary, to achieve the maximum percent identity. Terminal (5′ or 3′) or internal deletions, extensions or insertions into the nucleotide sequence shall not be construed as affecting identity.

In some embodiments, the polynucleotides comprise primers and probes that specifically hybridize to an HSV-1, HSV-2 and VZV target sequence, for example, the nucleic acid molecules having any one of the nucleic acid sequences set forth in SEQ ID NOs: 1-21 including analogues and/or derivatives of said nucleic acid sequences, and homologues thereof, that can specifically hybridize with an HSV-1, HSV-2 and VZV target sequence. As described below, polynucleotides find use as primers and/or probes to amplify or detect HSV-1, HSV-2 and VZV.

The polynucleotides can be prepared by a variety of techniques. For example, the polynucleotides can be prepared using solid-phase synthesis using commercially available equipment, such as that available from Applied Biosystems USA Inc. (Foster City, Calif), DuPont, (Wilmington, Del.), or Milligen (Bedford, Mass.). Modified polynucleotides, such as phosphorothioates and alkylated derivatives, can also be readily prepared (see, for example, U.S. Pat. Nos. 5,464,746; 5,424,414; and 4,948,882).

The polynucleotides can be employed directly as probes for the detection, or quantitation, or both, of HSV-1, HSV-2 and VZV nucleic acids in a test sample. The test sample is contacted with at least one of the polynucleotides under suitable hybridization conditions and the hybridization between the target sequence and at least one of the polynucleotides is then detected. Detection can be direct or indirect. In some embodiments, a hybrid between the probe and target is detected directly. In some embodiments, the hybrid is detected indirectly, for example, by detecting reaction byproducts generated by an enzymatic reaction that occurs in the presence of a duplex between a probe and the HSV-1, HSV-2 and VZV target.

The polynucleotides may incorporate one or more detectable labels. Detectable labels are molecules or moieties having a property or characteristic that can be detected directly or indirectly and are chosen such that the ability of the polynucleotide to hybridize with its target sequence is not adversely affected.

Detection labels have the same definition as “labels” previously defined and “capture labels” are typically used to separate extension products, and probes associated with any such products, from other amplification reactants. Specific binding members (as previously defined) are well suited for this purpose. Also, probes used according to this method may be blocked at their 3′ ends so that they are not extended under hybridization conditions. Methods for preventing extension of a probe are well known and are a matter of choice for one skilled in the art.

In cases where labels are employed to detect primer-amplified products, primer sequences optionally can be labeled with either a capture label or a detection label. In some embodiments, primers comprise a 3′ portion that hybridize to a HSV-1, HSV-2 and VZV target nucleic acid and a 5′ portion that introduces a non-HSV-1, HSV-2 and VZV sequence to extension products generated therefrom. Such 5′ portions may include a synthetic tag sequence for use, for example, in next-generation sequencing technologies e.g., Illumina, PacBio, ONT and the like.

In some embodiments, a probe is used to hybridize with the extension product or amplicon generated by the primer sequences, and typically hybridizes with a sequence that does not include the primer sequences. Similar to the primer sequence, the probe sequence can also be labeled with either a capture label or a detection label with the caveat that, in some embodiments, when the primer is labeled with a capture label, the probe is labeled with a detection label, and vice versa. Upon formation of the copy sequence/probe hybrids, the differential labels (i.e., capture and detection labels) on the copy sequence and probe sequence can be used to separate and detect such hybrids.

The polynucleotides are also suitable for use as capture probes in sandwich-type assays. The polynucleotide capture probe is attached to a solid support and brought into contact with a test sample under suitable hybridization conditions such that a probe:target hybrid is formed between the capture probe and any target nucleic acid present in the test sample. After one or more appropriate washing steps, the probe:target hybrid is detected, usually by means of a second “disclosure” probe or by a specific antibody that recognizes the hybrid molecule.

Embodiments also contemplate the use of the polynucleotides in modified nucleic acid hybridization assays. For example, U.S. Pat. No. 5,627,030 discloses a method to amplify the detection signal in a nucleic acid hybridization assay. In the disclosed assay, a first polynucleotide probe sequence is hybridized under suitable conditions to a target sequence, the probe:target hybrid is subsequently immunocaptured and immobilized. A second polynucleotide probe which contains many repeating sequence units is then hybridized to the probe component of the probe:target hybrid. Detection is achieved by hybridization of many labeled nucleic acid sequence probes, one to each of the repeating sequence units present in the second probe. The attachment of multiple labeled probes to the second probe thus amplifies the detection signal and increases the sensitivity of the assay.

### Amplification and Detection of HSV-1, HSV-2 and VZV Nucleotide Sequences

The polynucleotides can be used as primers or probes to amplify and/or detect HSV-1, HSV-2 and VZV in a test sample. The primer/probe sets provided herein comprise at least two primers and at least one probe. These primer/probe sets can be employed according to nucleic acid amplification techniques. Hence, the primers in any particular primer/probe set can be employed to amplify a target sequence. In most cases, the probe hybridizes to the copies of the target sequence generated by one or more of the primers and generally facilitates detecting any copies of the target sequence generated during the course of the amplification reaction. All of the primer/probe sets can be employed according to nucleic acid amplification procedures to specifically and sensitively detect HSV-1, HSV-2 and VZV when the appropriate primers and probes are combined. It is contemplated that the individual primers and probes of the primer/probe sets provided herein may alternatively be used in combination with primers and/or probes other than those described in the primer/probe sets provided herein. In some embodiments, two primer and probes sets are employed to detect two different HSV-1, HSV-2 and VZV target sequences.

Amplification procedures include, but are not limited to, polymerase chain reaction (PCR), TMA, rolling circle amplification, nucleic acid sequence-based amplification (NASBA), and strand displacement amplification (SDA). One skilled in the art will understand that for use in certain amplification techniques the primers may need to be modified, for example, for SDA the primer comprises additional nucleotides near its 5′ end that constitute a recognition site for a restriction endonuclease. Similarly, for NASBA the primer comprises additional nucleotides near the 5′ end that constitute an RNA polymerase promoter.

In some embodiments, certain criteria are taken into consideration when selecting a primer for an amplification reaction. For example, when a primer pair is required for the amplification reaction, the primers should be selected such that the likelihood of forming 3′ duplexes is minimized, and such that the melting temperatures (T_{M}) are sufficiently similar to optimize annealing to the target sequence and minimize the amount of non-specific annealing.

In some embodiments, the amplification methods comprises: (a) forming a reaction mixture comprising nucleic acid amplification reagents, at least one primer/probe set, and a test sample suspected of containing at least one target sequence and (b) subjecting the mixture to amplification conditions to generate at least one copy of a nucleic acid sequence complementary to the target sequence. Step (b) of the above methods can be repeated any suitable number of times (prior to step (c) in the detection method), e.g., by thermal cycling the reaction mixture between 10 and 100 times, typically between about 20 and about 60 times, more typically between about 25 and about 45 times.

Nucleic acid amplification reagents include but are not limited to, an enzyme having at least polymerase activity, enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

Amplification conditions are conditions that generally promote annealing and extension of one or more nucleic acid sequences.

Specific amplicons produced by amplification of target nucleic acid sequences using the polynucleotides, as described above, can be detected by a variety of methods. For example, one or more of the primers used in the amplification reactions may be labeled such that an amplicon can be directly detected by conventional techniques subsequent to the amplification reaction. Alternatively, a probe consisting of a labeled version of one of the primers used in the amplification reaction, or a third polynucleotide distinct from the primer sequences that has been labeled and is complementary to a region of the amplified sequence, can be added after the amplification reaction is complete. The mixture is then submitted to appropriate hybridization and wash conditions and the label is detected by conventional methods.

The amplification product produced as above can be detected during or subsequently to the amplification of the target sequence. Methods for detecting the amplification of a target sequence during amplification (e.g., real-time PCR) are outlined above, and described, for example, in U.S. Pat. No. 5,210,015. Alternatively, amplification products are hybridized to probes, then separated from other reaction components and detected using microparticles and labeled probes.

It will be readily appreciated that a procedure that allows both amplification and detection of target nucleic acid sequences to take place concurrently in a single unopened reaction vessel would be advantageous. Such a procedure avoids the risk of “carry-over” contamination in the post-amplification processing steps, and also facilitates high-throughput screening or assays and the adaptation of the procedure to automation. Furthermore, this type of procedure allows “real-time” monitoring of the amplification reaction as well as “end-point” monitoring. Examples of probe molecules that are particularly well-suited to this type of procedure include molecular beacon probes and TAQMAN probes. TAQMAN probes are generally dual-labeled fluorogenic nucleic acid probes composed of a polynucleotide complementary to the target sequence that is labeled at the 5′ terminus with a fluorophore and at the 3′ terminus with a quencher. In the free probe, the close proximity of the fluorophore and the quencher ensures that the fluorophore is internally quenched. During the extension phase of the amplification reaction, the probe is cleaved by the 5′ nuclease activity of the polymerase and the fluorophore is released. The released fluorophore can then fluoresce and thus produces a detectable signal.

In some embodiments, “molecular beacon” probes are employed. Molecular beacon probes are described, for example, in U.S. Pat. Nos. 6,150,097; 5,925,517 and 6,103,476 (herein incorporated by reference in their entireties). Molecular beacons are polynucleotide probes capable of forming a stem-loop (hairpin) structure. The loop is a single-stranded structure containing sequences complementary to the target sequence, whereas the stem typically is unrelated to the target sequence and self-hybridizes to form a double-stranded region. Nucleotides that are both complementary to the target sequence and that can self-hybridize may also form part of the stem region. Attached to one arm of the stem is a fluorophore moiety and to the other arm a quencher moiety. When the polynucleotide adopts a hairpin shape, the fluorophore and the quencher are in close proximity and the energy emitted by the fluorophore is thus absorbed by the quencher and given off as heat, resulting in internal quenching of the fluorophore. Upon binding of the polynucleotide to its target sequence, the fluorophore and the quencher become spatially separated and the fluorophore can fluoresce producing a detectable signal.

Examples of fluorophores that find use in the present invention include, but are not limited to, fluorescein and fluorescein derivatives such as a dihalo-(C₁ to C₈)dialkoxycarboxyfluorescein, 5-(2′-aminoethyl)aminonaphthalene-1-sulphonic acid (EDANS), coumarin and coumarin derivatives, Lucifer yellow, Texas red, tetramethylrhodamine, tetrachloro-6-carboxyfluoroscein, 5-carboxyrhodamine, cyanine dyes and the like. Quenchers include, but are not limited to, DABCYL, 4′-(4-dimethylaminophenylazo)benzoic acid (DABSYL), 4-dimethylaminophenylazophenyl-4′-maleimide (DABMI), tetramethylrhodamine, carboxytetramethylrhodamine (TAMRA), Black Hole Quencher (BHQ) dyes and the like.

In some embodiments, quantitative assays are employed. In some such embodiments, an internal standard is employed in the reaction. Such internal standards generally comprise a control target nucleic acid sequence and a control polynucleotide probe. The internal standard can optionally further include an additional pair of primers. The primary sequence of these control primers may be unrelated to the HSV-1, HSV-2 and VZV polynucleotides and specific for the control target nucleic acid sequence. Alternatively, no additional primer need be used if the control target sequence is designed such that it binds the HSV-1, HSV-2 and VZV primers. The amount of target nucleic acid in a test sample can be quantified using “end point” methods or “real time” methods.

In some embodiments, HSV-1, HSV-2 and VZV detection assays are provided as high-throughput assays. For high-throughput assays, reaction components are usually housed in a multi-container carrier or platform, such as a multi-well microtiter plate, which allows a plurality of assay reactions containing different test samples to be monitored in the same assay. In some embodiments, highly automated high-throughput assays are employed to increase the efficiency of the screening or assay process. Many high-throughput screening or assay systems are now available commercially, as are automation capabilities for many procedures such as sample and reagent pipetting, liquid dispensing, timed incubations, formatting samples into microarrays, microplate thermocycling and microplate readings in an appropriate detector, resulting in much faster throughput times. In some embodiments, reactions are performed in microfluidic devices (e.g., cards).

The polynucleotides, methods, and kits are useful in clinical or research settings for the detection and/or quantitation of HSV-1, HSV-2 and VZV nucleic acids. Thus, in these settings the polynucleotides can be used in assays to diagnose HSV-1, HSV-2 and VZV infection in a subject, or to monitor the quantity of an HSV-1, HSV-2 and VZV target nucleic acid sequence in a subject infected with HSV-1, HSV-2 and VZV. Monitoring the quantity of virus in a subject is particularly important in identifying or monitoring response to anti-viral therapy.

In some embodiments, the assays are amenable for use with automated real-time PCR detection system, such as the Abbott m2000 system or the Abbott Alinity m® system. Thus, in some embodiments, prior to conducting an assay, the samples are prepared for use with such systems. For example, in some embodiments, preparation of target DNA is performed using a magnetic microparticle-based technology (Abbott mSample Preparation SystemDNA). This can be performed using an Abbott m2000sp for automated sample preparation or using a manual sample preparation protocol. In some embodiments, an internal control (IC), positive control, and negative control are processed from the start of sample preparation to demonstrate that the process has proceeded correctly.

For amplification, in some embodiments, purified sample DNA and master mix are added to a 96-well PCR plate using an Abbott m2000sp instrument or manually. After addition, each plate is sealed and transferred to an Abbott m2000rt where PCR amplification is performed using DNA Polymerase.

In some embodiments, the presence of HSV-1, HSV-2 and VZV amplification products is detected during the annealing/extension step by measuring the real-time fluorescence signal of the HSV-1, HSV-2 and VZV probes. The presence of IC amplification products is detected by measuring the real-time fluorescence signal of the IC probe. In some embodiments, the HSV-1, HSV-2 and VZV and IC probes are single-stranded DNA oligonucleotides consisting of the target-specific binding sequence, a fluorescent moiety covalently linked to the 5′ end of the probe, and a quenching moiety covalently linked to the 3′ end of the probe. In the absence of the HSV-1, HSV-2 and VZV or IC target sequences, probe fluorescence is quenched. In the presence of HSV-1, HSV-2 and VZV or IC target sequences, the HSV-1, HSV-2 and VZV or IC probes specifically bind to their complementary sequences in the targets during the annealing/extension step, allowing fluorescent emission and detection. In some embodiments, the HSV-1, HSV-2 and VZV probes are labeled with different fluorescent dyes (e.g., C560 for HSV-1, FAM™ for HSV-2, Quasar® 705 for VZV target probes, and Quasar® 670 for IC), thus allowing the amplification products of HSV-1, HSV-2 and VZV and IC to be simultaneously detected in the same reaction.

In some embodiments, steps are taken to avoid nucleic acid contamination. For example, in some embodiments, contamination is minimized because: PCR amplification and oligonucleotide hybridization occur in a sealed reaction vessels; detection is carried out automatically without the need to open the reaction vessels (e.g., plate wells); aerosol barrier pipette tips are used for all pipetting; the pipette tips are discarded after use; and separate dedicated areas are used to perform the HSV-1, HSV-2 and VZV assay.

In some embodiments, the above reagents are provided in the form of a kit and/or system (e.g., systems comprising automated sample handling and assay instruments described herein). For example, in some embodiments, the kit and/or system comprises, consists essentially of, or consists of:
1. Alinity m HSV 1 & 2/VZV AMP Kit: Alinity m HSV 1 & 2/VZV AMP kit is comprised of 2 types of multi-well trays: Alinity m HSV 1 & 2/VZV AMP TRAY 1 and Alinity m HSV 1 & 2/VZV ACT TRAY 2.
   i. Each Alinity m HSV 1 & 2/VZV AMP TRAY 1 (individually packed in a foil pouch) contains 48 unit-dose liquid amplification reagent wells and 48 unit-dose liquid IC wells. One well of each is used per test. Amplification reagent wells consist of synthetic oligonucleotides, DNA polymerase, dNTPs and 0.15% ProClin© 950 in a buffered solution. Internal control (IC) wells consist of linearized plasmid DNA with unrelated IC sequences, and poly dA:dT in TE buffer containing, 0.15% ProClin© 950 as preservative.
   ii. Each Alinity m HSV 1 & 2/VZV ACT TRAY 2 (individually packed in a foil pouch) contains 48 unit-dose liquid activation reagent wells. One reagent well is used per test. Alinity m HSV 1 & 2/VZV ACT TRAY 2 contains unit-dose activation reagents (KCl, MgCl2, TMAC and ProClin® 950).
2. Alinity m HSV 1 & 2/VZV CTRL Kit: Alinity m HSV 1 & 2/VZV CTRL Kit consists of negative controls and positive controls, each supplied as liquid in single-use tubes.
   i. Alinity m HSV 1 & 2/VZV Negative CTRL (List No. 9N61Z) contains a buffer solution.
   ii. Alinity m HSV 1 & 2/VZV Positive CTRL (List No. 9N61W) contains a mixture of linearized plasmid DNA containing HSV-1, HSV-2 and VZV DNA sequences in a buffer solution.

In some embodiments, the Alinity m® HSV 1 & 2/VZV AMP kit and/or system comprises, consists essentially of, or consists of 2 types of multi-well trays i.e., Alinity m® HSV 1 & 2/VZV AMP TRAY 1 and Alinity m® HSV 1 & 2/VZV ACT TRAY 2. The Alinity m® HSV 1 & 2/VZV AMP TRAY 1 is individually packed in a foil pouch and contains 48 unit-dose liquid amplification reagent wells, and 48 unit-dose liquid internal control (IC) wells. One well of each is used per test. Amplification reagent wells comprise synthetic oligonucleotides, DNA polymerase, dNTPs and 0.15% ProClin® 950 in a buffered solution. Internal control (IC) wells comprise linearized plasmid DNA with unrelated IC sequences, and poly dA:dT in TE buffer containing 0.15% ProClin® 950 as preservative. The Alinity m® HSV 1 & 2/VZV ACT TRAY 2 is individually packed in a foil pouch and contains 48 unit-dose liquid activation reagent wells. One reagent well is used per test. In some embodiments, the Alinity m® HSV 1 & 2/VZV ACT TRAY 2 comprises unit-dose activation reagents, for example, KCl, MgCl2, TMAC and ProClin® 95. In some embodiments, the Alinity m® HSV 1 & 2/VZV CTRL Kit comprises negative controls and positive controls supplied as liquid in single-use tubes. The Alinity m® HSV 1 & 2/VZV Negative CTRL kit comprises a buffer solution. The Alinity m® HSV 1 & 2/VZV Positive CTRL kit comprises a mixture of linearized plasmid DNA containing HSV-1, HSV-2 and VZV DNA sequences in a buffer solution.

In some embodiments, all forms of HSV-1, HSV-2 and VZV are detected (e.g., the primers and probes are selected to identify all HSV-1, HSV-2 and VZV nucleic target sequences that might be present in a sample). In some embodiments, specific HSV-1, HSV-2 and VZV sequences are detected.

### EXAMPLES

The following examples are for illustrative purposes only and should not be construed to limit the scope of this invention in any way.

### Example 1—Exemplary Assay Workflow

This example describes an approach to conducting real-time PCR to detect HSV-1, HSV-2 and VZV in a sample. In some embodiments, real-time PCR methods comprise or consist of the following steps:
1. Sample preparation in which DNA is extracted from the samples using reagents; sample preparation is performed using the automated Abbott Alinity m® system (Abbott Molecular), or manually;
2. PCR assembly in which purified samples and assay PCR components are added together in a reaction vessel performed using the Alinity m® system or manually;
3. Transfer of the reaction vessel to an Amplification detection module on Alinity m® system.
4. Amplification and detection of PCR products using the automated Alinity m® system. Subject results are automatically reported on the Alinity m® system workstation.

### Example 2—HSV-1, HSV-2 and VZV Triplex Assay Tested with Cultured Virus

A multiplex (e.g., triplex) real time polymerase chain reaction (PCR) assay was configured and tested for direct qualitative detection and differentiation of HSV-1, HSV-2 and VZV DNA from cultured virus samples. In some embodiments, the assay finds use as an aid in the diagnosis of HSV-1, HSV-2 and/or VZV infections in symptomatic and/or asymptomatic patients. In some embodiments, the assay is configured for use with the automated Abbott Alinity m® system. In some embodiments, the assay finds use with other real time PCR instruments, and with other samples, for example, cerebrospinal fluid (CSF) samples. To test the performance of the Alinity m® system HSV 1, HSV 2 and VZV assay, viral panel members (PMs) containing HSV-1, HSV-2 or VZV viral particles were tested in comparison with other methods at other sites comprising: Simplexa HSV 1 & 2 Direct Kit (DIASORIN MOLECULAR, Cypress, CA) at Northshore University Health center, Evanston, IL; Artus HSV-1/2 RG PCR Kit (QIAGEN, Hilden, Germany) at Quest Diagnostics, Lewisville, TX; Luminex ARIES HSV 1 & 2 (Luminex, Austin, TX) at Tricore Reference Labs, Albuquerque, NM; Solana HSV/VZV (Quidel, San Diego, CA) at Tricore Reference Labs, Albuquerque, NM; Simplexa VZV Direct kit (DIASORIN MOLECULAR, Cypress, CA) at Northshore University Health Center, Evanston, IL; Artus VZV QS-RGQ Kit (QIAGEN, Hilden, Germany) at Quest Diagnostics, Lewisville, TX; HSV 1 & 2 on COBAS® 4800 (Roche Diagnostics, Indianapolis, IN) at BocaBiolistics, Pompano Beach, FL; Aptima HSV 1 & 2 testing on PANTHER® (Hologic, Santa Clara, CA) at BocaBiolistics, Pompano Beach, FL. The samples were further tested with the Alinity m® HSV 1 & 2/VZV assay at Abbott, Des Plaines. IL

### HSV-1, HSV-2 and VZV Target Panel Preparation

A panel consisting of different levels of HSV-1, HSV-2 and VZV DNA was prepared with respective viral cultures diluted in different transport media. (See Table 5, below.) Virus and strains used in the panel preparation were: HSV-1 MacIntyre (ATCC, Manassas, VA), HSV-1 HF (ATCC, Manassas, VA), HSV-2 MS (ATCC, Manassas, VA), HSV-2 G (ATCC, Manassas, VA), VZV 82 (ZeptoMetrix, Buffalo, NY) and VZV Ellen (ZeptoMetrix, Buffalo, NY). For HSV-1, HSV-1 MacIntyre and HSV-1 HF strains were tested. For HSV-2, HSV-2 MS and HSV-2 G were tested. For VZV, VZV 82 and VZV Ellen were tested. The strains were diluted in BD Universal Viral Transport media (BD-UVT) (BD, Franklin Lakes, NJ) to generate 7 concentrations for each HSV-1 and HSV-2 analyte panel member. For HSV-1, the concentrations were: 1024, 256, 64, 16, 4, 1, and 0.5 TCID₅₀/mL. For HSV-2, the concentrations were: 4096, 1024, 256, 64, 16, 4, 1, and 0.5 TCID₅₀/mL. For VZV panel members, the concentrations were 51200, 12800, 3200, 800, 200, 50, 25, 12.5, 6.25, 3.125, 1.56 and copies/mL. The panel members were dispensed into 1 mL aliquots and stored at −70° C. For each analyte, a negative control panel member, consisting of only BD-UVT, was also dispensed into 1 mL aliquots and stored at −70° C.

### Primers and Probe Design

Primers and probes were designed for amplification of genomic regions from HSV-1, HSV-2 and VZV. The assay formulation comprises 2 sets of primers/probe sets (i.e., dual target primer/probe sets) for each of 3 analytes i.e., HSV-1, HSV-2 and VZV. The gene/targets for the dual target design for HSV-1 are Glycoprotein D (US6) and Unique Long Region (UL) 1. For HSV-2, the gene/targets for the dual target design are UL1+UL18. Open Reading Frame (ORF) 10 and ORF 21 were used as the dual gene/targets for VZV. The primer and probe sequences for targets of each virus are provided in Table 1. Forward and Reverse primers for each gene and target anneal to the specific target sequence and direct the amplification of the target. The probes then specifically bind to their respective amplicon and facilitate detection of HSV-1, HSV-2 and/or VZV. The probes have a fluorescent moiety that is covalently linked to the 5′ end and have a quencher molecule at the 3′ end. In the absence of target sequences, probe fluorescence is quenched. In the presence of target sequences, hybridization to complementary sequences separates the fluorophore and the quencher and allows fluorescent emission and detection. The probe for each analyte is linked to a specific dye which allows detection of each analyte in a specific lamination channel resulting in differentiation of all 4 targets (HSV-1, HSV-2, VZV and IC).

TABLE 1
Primer pair and probe sequences of the Alinity m HSV-1, HSV-2 and VZV assay

| Target | Oligonucleotide | Sequence and label (5′→3′) |
|---|---|---|
| HSV-1 target 1 | HSV-1 A Fwd Primer | ACCATCGCTTGGTTTCGG (SEQ ID NO: 1) |
| (US-6) | HSV-1 A Rev primer | CCCCAGAGACTTGTTGTAGGA (SEQ ID NO: 2) |
| | HSV-1 A Probe | C560-AGGCAACTGTGCTATCCCCA-BHQ1 dT |
| | | (SEQ ID NO: 3) |
| HSV-1 target 2 | HSV-1 B Fwd Primer | GAAACAGAAACGCGCTTG (SEQ ID NO: 4) |
| (UL-1) | HSV-1 B Rev primer | GTCCGACGTGGCGATGATG (SEQ ID NO: 5) |
| | HSV-1 B Probe | C560-TGTAGGGCGACAGGATTTGG-BHQ1 Dt |
| | | (SEQ ID NO: 6) |
| HSV-2 target 1 | HSV-2 A Fwd Primer | CGGACGATGTTTCTTGG (SEQ ID NO: 7) |
| (UL-1) | HSV-2 A Rev primer | GCAGTGATAGCGAAGAAATATTC (SEQ ID NO: 8) |
| | HSV-2 A Probe | FAM-TACGAGGCCCCGTCCGTTATTG-BHQ1 Dt |
| | | (SEQ ID NO: 9) |
| HSV-2 target 2 | HSV-2 B Fwd Primer | GATGAGGGTGGTTCCTTCG (SEQ ID NO: 10) |
| (UL-18) | HSV-2 B Rev primer | CCCGTCTCTCGCTCGTGG (SEQ ID NO: 11) |
| | HSV-2 B Probe | FAM-TGTTTACGGAGGCGAGTTGCTG-BHQ1 Dt |
| | | (SEQ ID NO: 12) |
| VZV target 1 | VZV A Fwd Primer | AAACCACTAACAGCGTCTGCC (SEQ ID NO: 13) |
| (ORF10) | VZV A Rev primer | ACCGAAAATGAGGGTTGTTGAAC (SEQ ID NO: 14) |
| | VZV A Probe | Q705-TTG AGG GAA ATA AAT TAC CGC CGC C-BHQ2 dT |
| | | (SEQ ID NO: 15) |
| VZV target 2 | VZV B Fwd Primer | CGGAGGCTGCTTTCACGG (SEQ ID NO: 16) |
| (ORF21) | VZV B Rev primer | GGCCGATAAACGATGTAACGT (SEQ ID NO: 17) |
| | VZV B Probe | Q705-TAGACTGGACGATAGAGGAGCCAGG-BHQ2 dT |
| | | (SEQ ID NO: 18) |
| IC | IC Fwd Primer | CTACAGCAGAGTTGGCAGCTTCACTTTC |
| | | (SEQ ID NO: 19) |
| | IC Rev Primer | ACAAATTTGGAAGCCATCCATCA (SEQ ID NO: 20) |
| | IC Probe | Q670-ACGAGTTCATGAGGGCAGGCCGCT-BHQ2 dT |
| | | (SEQ ID NO: 21) |

In some embodiments, probe/dye combinations of Table 1 are reconfigured to achieve a variety of detection outputs. In some embodiments, the primer pairs and probes of Table 1 are configured to achieve detection outputs in a multiplex format, in simultaneous tests, in a single reaction vessel or container, in shared reagents and concentrations of reagents, under shared reaction and amplification conditions. The probe sequences of Table 1 are derived from one strand of the viral genome. In some embodiments, the complementary sequence may be used. In some embodiments, label at the opposite end from the dye may be any moiety that can effectively quench the fluorescent dye and allow effective association of these probes to their target sequences. In some embodiments, the dye is labeled at the 5′ end of the probe with the quencher at the 3′ end. In some embodiments, the dye is labeled at the 3′ end with the quencher at the 5′ end. In some embodiments, the primers and/or probes are be modified to comprise one or more binding enhancers, for example, mgb or pdU/pdC.

### PCR Formulation

The multiplex assay comprises a single reaction mixture for simultaneous detection of DNA from HSV-1, HSV-2, VZV and IC. The PCR reaction contained sample DNA and PCR mastermix. The PCR mastermix comprised primers and probes for HSV-1, HSV-2, VZV and IC, 12x Alinity® Buffer, dNTPs, DNA polymerase, C610 reference dye, Gelatin, Proclin, KCl, TMAC (Tetramethylammonium chloride), MgCl2 and water. KCl, TMAC and MgCl2 were components of the activation reagent. The composition of oligonucleotide mixture and activation reagent are provided in Table 2, and 3.

TABLE 2
Oligonucleotide mastermix composition

| Component | Concentration | Unit |
|---|---|---|
| HSV-1 A Fwd Primer | 0.300 | uM |
| HSV-1 A Rev primer | 0.300 | uM |
| HSV-1 A Probe | 0.150 | uM |
| HSV-1 B Fwd Primer | 0.300 | uM |
| HSV-1 B Rev primer | 0.300 | uM |
| HSV-1 B Probe | 0.150 | uM |
| HSV-2 A Fwd Primer | 0.300 | uM |
| HSV-2 A Rev primer | 0.300 | uM |
| HSV-2 A Probe | 0.150 | uM |
| HSV-2 B Fwd Primer | 0.300 | uM |
| HSV-2 B Rev primer | 0.300 | uM |
| HSV-2 B Probe | 0.150 | uM |
| VZV A Fwd Primer | 0.200 | uM |
| VZV A Rev primer | 0.200 | uM |
| VZV A Probe | 0.075 | uM |
| VZV B Fwd Primer | 0.200 | uM |
| VZV B Rev primer | 0.200 | uM |
| VZV B Probe | 0.075 | uM |
| 12X Alinity ® Buffer | 1 | X |
| C610 Reference Dye | 0.015 | uM |
| dNTP | 0.800 | mM |
| DNA Polymerase | 8.8 | Units |
| Gelatin | 0.4 | % |
| Proclin 950 | 0.0375 | % |
| Water (to a final volume of 10 uL | NA | uL |
| per reaction) | | |

TABLE 3
Activation reagent composition

| Component | Concentration | Unit |
|---|---|---|
| KCl | 30 | mM |
| TMAC (Tetramethylammonium chloride) | 75 | mM |
| MgCl2 | 8 | mM |
| Water (to a final volume of 5 uL per | NA | uL |
| reaction) | | |

### PCR Reaction

On completion of the sample preparation process, the purified nucleic acid eluate, unit-dose Alinity m® HSV 1, HSV-2 and VZV amplification reagents, and unit-dose activation reagents were combined by the Alinity m® System in a PCR reaction vessel. A layer of Alinity m® Vapor Barrier Solution was then automatically added to the reaction vessel. The reaction vessel was then capped and transferred to a thermal cycling position in the Assay Processing Unit (APU). for amplification of specified targets and detection.

### Cycling Conditions

During PCR, the reaction temperature rose to a temperature that dissociated double-stranded DNA. As the reaction temperature was subsequently lowered, HSV-1, HSV-2 and VZV primers annealed to their respective DNA strands and were extended by the Platinum II Taq DNA polymerase. During successive rounds of thermal cycling, amplification products dissociated to single strands at high temperature, followed by primer annealing and extension as the temperature was lowered. Exponential amplification was achieved through repeated cycling between high and low temperatures. Amplification of the dual targets for the 4 assay analytes (i.e., HSV-1, HSV-2, VZV and IC) took place simultaneously in the same reaction. During the primer annealing/extension step, a read step was performed to allow real-time fluorescent detection of amplification products as the probes annealed to their respective targets. The probes have a fluorescent moiety that is covalently linked to the 5′ end with a quencher molecule at the 3′ end. In the absence of target sequences, probe fluorescence was quenched. In the presence of target sequences, hybridization to complementary sequences separated the fluorophore and the quencher and allowed fluorescent emission and detection. PCR cycling conditions are provided in Table 4.

TABLE 4
PCR cycling conditions

| | | | | | | Overshoot | Ramp |
|---|---|---|---|---|---|---|---|
| | # of | | Temp. | Time | Overshoot | Time | Rate |
| Stage | Cycles | Step | (° C.) | (sec) | (° C.) | (sec) | (° C./sec) |
| 1 | 1 | 1 | 95 | 326.63 | 10 | 6.0 | 5.5 |
| 2 | 5 | 1 | 97 | 7 | 10 | 6.0 | 5.5 |
| | | 2 | 63 | 34.62 | 10 | 4.0 | 5.5 |
| 3 | 37 | 1 | 97 | 7 | 10 | 6.0 | 5.5 |
| | | 2 | 59 | 33.18 | 6 | 4.0 | 5.5 |

| | | | |
|---|---|---|---|
| Read Protocol | Read Start = 46 | Read Gap = 4 | Read Count = 37 |

| | |
|---|---|
| Global Overshoot = 0° C. | Global Overshoot Duration = 0 sec |
| Global Ramp Rate = 5° C./sec | Global Read Temperature = 59° C. |

### Results

Results are reported in cycle number (CN) for each target (HSV-1, HSV-2, VZV, and IC) based on the threshold cycle (Ct) at which the fluorescent signal surpasses a threshold to indicate detection of the target nucleic acids. Ct is inversely proportional to the concentration of each analyte present.

Accordingly, in some embodiments the present invention provides a diagnostic method comprising the steps of:
1. Preparing a reaction mixture of the 21 oligonucleotides provided in Table 1, and PCR reagents such as dNTP, 12x Alinity m® Buffer, Fish Gelatin, C610 Reference Dye and Platinum II Polymerase to prepare a mastermix.
2. Mixing the reaction mixture with divalent ion as activation reagent.
3. DNA extraction from HSV-1, HSV-2 or VZV virus.
4. Mixing eluted DNA with mastermix and activation reagent to amplify HSV-1 US6, HSV-1 UL1, HSV-2 UL1, HSV-2 UL18, VZV ORF10 and VZV ORF21 sequences from their respective virus genomes.
5. Detection of each analyte with a separate fluorophore to identify and distinguish HSV-1, HSV-2 and VZV targets.

### Comparison Testing

The cultured samples were also forwarded to laboratories for testing with following comparison assays:
1. Simplexa® HSV 1 & 2 Direct Kit at Northshore University Health Center
2. Artus® HSV-1/2 RG PCR Kit at Quest Diagnostics
3. Luminex® ARIES HSV 1 & 2 at Tricore Reference Labs
4. Solana® HSV/VZV at Tricore Reference Labs
5. Simplexa® VZV Direct kit at Northshore University Health Center
6. Artus® VZV QS-RGQ Kit at Quest Diagnostics
7. Cobas® HSV 1 and 2 Test on COBAS® 4800 System at BocaBliolistics
8. Aptima Herpes Simplex Viruses 1 & 2 Assay on PANTHER® at BocaBliolisties

The identical HSV-1, HSV-2 and VZV panels were tested on Alinity m® System with the methods described in sections above.

### Comparative Results in RD-UVT (UTM) Media

Panel members prepared in BD-UVT (UTM) were tested with Simplexa®, ARTUS®, Luminex® and Quidel/Solana® assays using respective assay package inserts or instructions for use. Panel members also tested with Alinity m® System HSV-1, HSV-2 and VZV assay. Assay-specific comparative data are provided in Table 5.

TABLE 5
Comparative HSV-1, HSV-2 and VZV assay results

| | | | | | | | | | Luminex |
|---|---|---|---|---|---|---|---|---|---|
| | | Alinity ® | | Simplexa ® | | Quest (ARTUS) ® | | Solana(Quidel) ® | (ARIES) ® |
| | | Mean | Detection | Mean | Detection | Mean | Detection | Detection | Detection |
| Strain | Titer | Ct | (n/N) | Ct | (n/N) | Ct | (n/N) | (n/N) | (n/N) |
| HSV- | 1024 | 19.11 | 3/3 | 30.80 | 2/2 | 27.43 | 3/3 | 2/2 | 3/3 |
| 1_MacIntyre | 256 | 21.39 | 3/3 | 33.65 | 2/2 | 29.84 | 3/3 | 1/2 | 3/3 |
| | 64 | 24.53 | 3/3 | 36.05 | 2/2 | 32.35 | 3/3 | 2/2 | 3/3 |
| | 16 | 25.22 | 3/3 | 35.75 | 2/2 | 36.92 | 2/3 | 0/2 | 1/3 |
| | 4 | 26.61 | 3/3 | | 0/2 | 37.68 | 2/3 | 0/2 | 0/3 |
| | 1 | 31.16 | 3/3 | | 0/2 | | 0/3 | 0/2 | 0/3 |
| | 0.5 | 32.43 | 2/3 | | 0/3 | | 0/3 | 0/2 | 0/3 |
| HSV- | 1024 | 19.22 | 3/3 | 32.10 | 2/2 | 29.85 | 3/3 | 2/2 | 3/3 |
| 1_HF | 256 | 23.01 | 3/3 | 33.05 | 2/2 | 32.18 | 3/3 | 0/2 | 3/3 |
| | 64 | 26.50 | 3/3 | 35.10 | 2/2 | 33.04 | 3/3 | 0/2 | 3/3 |
| | 16 | 28.11 | 3/3 | | 0/2 | 36.35 | 1/3 | 0/2 | 1/3 |
| | 4 | 30.21 | 3/3 | | 0/2 | 36.60 | 2/3 | 0/2 | 0/3 |
| | 1 | 30.42 | 3/3 | 37.70 | 1/2 | 37.82 | 1/3 | 0/2 | 0/3 |
| | 0.5 | 32.76 | 3/3 | | 0/2 | 32.51 | 1/3 | 0/2 | 1/3 |
| HSV- | 4096 | 19.11 | 3/3 | 34.50 | 2/2 | 24.56 | 3/3 | 2/2 | 3/3 |
| 2_MS | 1024 | 20.85 | 3/3 | 29.85 | 2/2 | 27.03 | 3/3 | 2/2 | 3/3 |
| | 256 | 22.90 | 3/3 | 31.75 | 2/2 | 28.73 | 3/3 | 2/2 | 3/3 |
| | 64 | 25.54 | 3/3 | 34.25 | 2/2 | 30.49 | 3/3 | 1/2 | 3/3 |
| | 16 | 27.18 | 3/3 | 36.60 | 2/2 | 31.39 | 3/3 | 0/2 | 3/3 |
| | 8 | 28.15 | 3/3 | | | 34.22 | 3/3 | | |
| | 4 | 29.58 | 3/3 | 38.75 | 2/2 | 35.03 | 2/3 | 0/2 | 2/3 |
| | 2 | 30.53 | 3/3 | 38.30 | 1/2 | 34.53 | 2/3 | 2/2 | 2/3 |
| | 1 | 31.33 | 3/3 | 39.70 | 1/3 | | | | |
| | 0.5 | 32.58 | 3/3 | 38.93 | 3/3 | | | | |
| HSV- | 4096 | 19.30 | 3/3 | 28.05 | 2/2 | 24.65 | 2/2 | 1/1 | 3/3 |
| 2_G | 1024 | 20.72 | 3/3 | 30.35 | 2/2 | 26.13 | 2/2 | 1/1 | 3/3 |
| | 256 | 22.78 | 3/3 | 31.45 | 2/2 | 27.82 | 3/3 | 1/1 | 3/3 |
| | 64 | 26.21 | 3/3 | 34.35 | 2/2 | 31.7 | 3/3 | 1/1 | 3/3 |
| | 32 | 26.53 | 3/3 | | | 31.91 | 3/3 | | |
| | 16 | 28.95 | 3/3 | 37.30 | 2/2 | 31.48 | 2/3 | 0/2 | 3/3 |
| | 4 | 32.03 | 3/3 | 38.30 | 2/2 | 37.02 | 3/3 | 0/2 | 0/3 |
| | 2 | 30.29 | 3/3 | 27.40 | 1/2 | | 0/3 | 0/2 | 1/3 |
| | 2 | | | 40.30 | 1/3 | | | | |
| | 1 | 28.43 | 3/3 | 39.80 | 1/3 | | | | |
| | 0.5 | 29.43 | 3/3 | | 0/3 | | | | |
| VZV_82 | 51200 | 19.01 | 3/3 | 28.65 | 2/2 | 23.28 | 3/3 | 1/1 | |
| | 12800 | 20.83 | 3/3 | 30.65 | 2/2 | 25.55 | 3/3 | 1/1 | |
| | 3200 | 22.66 | 3/3 | 31.80 | 2/2 | 27.39 | 3/3 | 1/1 | |
| | 800 | 24.93 | 3/3 | 34.75 | 2/2 | 29.58 | 3/3 | 1/1 | |
| | 200 | 27.55 | 3/3 | 37.45 | 2/2 | 31.87 | 3/3 | 1/1 | |
| | 50 | 29.07 | 3/3 | 39.10 | 2/2 | 33.08 | 3/3 | 0/1 | |
| | 25 | 30.37 | 3/3 | 39.10 | 1/2 | 34.54 | 3/3 | 0/1 | |
| | 12.5 | 31.94 | 3/3 | | | 36.24 | 3/3 | | |
| | 6.25 | 33.02 | 3/3 | | | 36.66 | 2/3 | | |
| | 3.125 | 33.35 | 3/3 | | | | 0/3 | | |
| | 1.56 | 33.96 | 2/3 | | | 37.78 | 1/3 | | |
| VZV_Ellen | 51200 | 21.20 | 3/3 | 31.70 | 2/2 | 25.06 | 3/3 | 2/2 | |
| | 12800 | 22.82 | 3/3 | 33.80 | 2/2 | 27.09 | 3/3 | 1/2 | |
| | 3200 | 24.75 | 3/3 | 35.95 | 2/2 | 28.90 | 3/3 | 1/2 | |
| | 800 | 26.77 | 3/3 | 37.85 | 2/2 | 30.75 | 3/3 | 0/2 | |
| | 200 | 28.56 | 3/3 | 39.35 | 2/2 | 32.92 | 3/3 | 0/2 | |
| | 50 | 30.30 | 3/3 | 41.60 | 1/2 | 35.55 | 3/3 | 0/1 | |
| | 25 | 31.96 | 3/3 | | 0/2 | 35.79 | 3/3 | 0/1 | |
| | 12.5 | 34.26 | 3/3 | | | | 0/3 | | |
| | 6.25 | 33.13 | 3/3 | | | 35.46 | 2/3 | | |
| | 3.125 | 35.32 | 2/3 | | | | 0/3 | | |

Panel members prepared in MSwab media (Roche Diagnostics, Indianapolis, IN) were tested with the Cobas® HSV 1 and 2 Test. Panel members prepared in Aptima Multitest Swab Specimen Collection Kit (Hologic, Santa Clara, CA) were tested with Aptima Herpes Simplex Viruses 1 & 2 Assay. The panel members were also tested with the Alinity m® HSV/VZV assay. Results for HSV-1 are provided in Table 6, and results for HSV-2 are provided in Table 7.

TABLE 6
Comparative HSV-1 assay results

| | | | | | | Alinity ®with |
|---|---|---|---|---|---|---|
| | | | PANTHER ® | | | Aptima |
| | | | Results | Results | Results | Buffer |
| Sample | | Level | HSV 1 | HSV 1 | HSV 1 | Results |
| ID | Analyte | (TCID50/mL) | REP 1 | REP 2 | REP 3 | HSV 1 |
| PM1 | HSV-1 | 0 | NEG | NEG | NEG | 0/3 |
| PM2 | HSV-1 | 256 | POS | POS | POS | 3/3 |
| PM3 | HSV-1 | 64 | POS | POS | POS | 3/3 |
| PM4 | HSV-1 | 16 | POS | POS | POS | 3/3 |
| PM5 | HSV-1 | 4 | NEG | NEG | NEG | 3/3 |
| PM6 | HSV-1 | 2 | NEG | NEG | NEG | 3/3 |
| PM7 | HSV-1 | 1 | NEG | NEG | NEG | 3/3 |
| PM8 | HSV-1 | 0.5 | NEG | NEG | NEG | 3/3 |
| PM9 | HSV-1 | 0.25 | NEG | NEG | NEG | 3/3 |
| PM10 | HSV-1 | 0.125 | NEG | NEG | NEG | 3/3 |

TABLE 7
Comparative HSV-2 assay results

| | | | | | | Alinity ® |
|---|---|---|---|---|---|---|
| | | | | | | with |
| | | | PANTHER ® | | | Aptima |
| | | | Results | Results | Results | Buffer |
| Sample | | Level | HSV 2 | HSV 2 | HSV 2 | Results |
| ID | Analyte | (TCID50/mL) | REP 1 | REP 2 | REP 3 | HSV 2 |
| PM11 | HSV-2 | 64 | POS | POS | POS | 3/3 |
| PM12 | HSV-2 | 16 | POS | POS | POS | 3/3 |
| PM13 | HSV-2 | 4 | POS | POS | POS | 3/3 |
| PM14 | HSV-2 | 2 | POS | POS | POS | 3/3 |
| PM15 | HSV-2 | 1 | POS | POS | POS | 3/3 |
| PM16 | HSV-2 | 0.5 | POS | POS | POS | 3/3 |
| PM17 | HSV-2 | 0.25 | POS | NEG | NEG | 3/3 |
| PM18 | HSV-2 | 0.125 | POS | POS | POS | 3/3 |
| PM19 | HSV-2 | 0.0625 | NEG | NEG | POS | 1/3 |
| PM20 | HSV-2 | 0 | NEG | NEG | NEG | 0/3 |

Table cells highlighted in green show detection for the respective analyte. Cells highlighted in grey were not tested, or the data were not obtained from the comparative assays due to invalid replicates. For HSV-1 MacIntyre and HSV-1 HF targets, the Alinity m® assay detected 100% replicates at 1 and 0.5 TCID₅₀/mL levels, respectively. For HSV-2 MS and HSV-2 G targets, the Alinity m® assay detected 100% replicates at 0.5 TCID₅₀/mL. For VZV 82 and VZV Ellen, the Alinity m® assay detected 100% replicates at 3.125 and 6.25 copies/mL levels respectively.

These data indicate that performance with the multiplex Alinity m® assay is equivalent or superior in comparison to conventional assays in precision, with additional advantages provided by the Alinity m® test format and platform in throughput, cost, sensitivity and specificity.

### Example 3—HSV-1, HSV-2 and VZV Triplex Assay Tested with Patient Samples

In some embodiments, the Alinity m® HSV 1 & 2/VZV assay is a multiplex real time Polymerase Chain Reaction (PCR) assay designed for direct qualitative detection and differentiation of HSV-1, HSV-2 and VZV DNA from cutaneous or mucocutaneous lesion specimens. To evaluate assay performance on patient samples, swab lesion clinical specimens collected in viral transport media were tested with Alinity m® HSV 1 & 2/VZV assay.

### Samples

Clinical specimens were obtained from commercial vendors (Trina Bioreactives, Switzerland; Discovery Life Sciences, Huntsville, AL). The specimens were acquired from patients and frozen prior to testing with the Alinity m® assay. A subset of specimens were never frozen prior to Alinity m® testing, and were tested as “fresh” (i.e., never frozen) samples. The fresh samples were collected from patients and not frozen at any time during storage. An aliquot of each of the samples was tested.

### Sample Preparation

Lesion swab samples were loaded on the Alinity m® System, and the sample preparation process was initiated. The purpose of sample preparation is to extract and concentrate nucleic acid for subsequent PCR amplification, and to remove PCR inhibitors from the resulting extract. The sample preparation protocol was performed within a disposable multi-well sample preparation cartridge loaded into one of the Assay Processing Units (APUs) on the Alinity m® System. Samples (i.e., specimen and/or control samples) and magnetic microparticles were pipetted by the instrument into an Integrated Reaction Unit (IRU) well containing Alinity m® Lysis Solution. An Internal Control (IC) sample was introduced into each sample at the beginning of the sample preparation process to assure that the process was completed correctly for each specimen and control sample. The conditions of the lysis step facilitate lysis of HSV-1, HSV-2 and VZV viral particles and denaturation of proteins. The lysis conditions also promote nucleic acid binding to the magnetic microparticles. At the conclusion of the lysis step, magnetic microparticles with bound sample nucleic acids were captured by a magnetic plunger sheathed with a disposable plastic sleeve. The magnetic microparticles were then successively transferred to wells within the IRU containing a series of wash solutions. After the wash steps were completed, the magnetic microparticles were captured by the plunger magnet, and transferred to an elution well within the IRU where the purified nucleic acid was eluted from the microparticles into Alinity m® Elution Buffer 2.

### Sample Testing

Testing was performed using Alinity m® HSV 1 & 2/VZV mastermix on an Alinity m® instrument with an Alinity m® HSV 1 & 2/VZV application specification file that contained software for sample processing and amplification of the targets (HSV-1, HSV-2, VZV, IC).

### Data Analysis

Results from were analyzed for cycle number (CN) for each target (HSV-1, HSV-2, VZV, and IC). The CN value is based on threshold cycle (Ct) at which the fluorescent signal surpasses a threshold to indicate the detection target nucleic acids.

### Experimental Results—Frozen Samples

Data from 91 frozen positive samples are shown in Table 8. The data comprise results from 30 HSV-1 positive samples that were positive for HSV-1 by both vendor results and by Alinity m® assay results. Results from 29 HSV-2 positive samples are also tabulated that were positive by both vendor results and Alinity m® assay. Data also comprise results from 32 VZV positive samples that were positive by both vendor results and by Alinity m® assay. Two of the VZV positive samples were dual positive for HSV. One was dual positive for VZV and HSV-1, and another sample was dual positive for VZV and HSV-2.

TABLE 8
Frozen sample data

| | | | Vendor | HSV- | HSV- | | | Alinity m ® |
|---|---|---|---|---|---|---|---|---|
| | Sample | Vendor | Result | 1_CT | 2_CT | VZV_CT | IC_CT | Result |
| | Negative | NA | NA | −1 | −1 | −1 | 21.67 | NA |
| | Control | | | | | | | |
| | Positive | NA | NA | 23.52 | 21.93 | 23.78 | 21.26 | NA |
| | Control | | | | | | | |
| 1 | Sample | Trina | HSV-1 pos. | 16.29 | −1 | −1 | 21.96 | HSV-1 positive |
| 2 | Sample | Trina | HSV-1 pos. | 14.60 | −1 | −1 | 20.45 | HSV-1 positive |
| 3 | Sample | Trina | HSV-1 pos. | 8.64 | −1 | −1 | 21.35 | HSV-1 positive |
| 4 | Sample | Trina | HSV-1 pos. | 15.67 | −1 | −1 | 20.40 | HSV-1 positive |
| 5 | Sample | Trina | HSV-1 pos. | 12.50 | −1 | −1 | 20.31 | HSV-1 positive |
| 6 | Sample | Trina | HSV-1 pos. | 15.44 | −1 | −1 | 21.13 | HSV-1 positive |
| 7 | Sample | Trina | HSV-1 pos. | 24.56 | −1 | −1 | 20.22 | HSV-1 positive |
| 8 | Sample | Trina | HSV-1 pos. | 17.00 | −1 | −1 | 20.02 | HSV-1 positive |
| 9 | Sample | Trina | HSV-1 pos. | 9.59 | −1 | −1 | 20.41 | HSV-1 positive |
| 10 | Sample | Trina | HSV-1 pos. | 18.36 | −1 | −1 | 20.50 | HSV-1 positive |
| 11 | Sample | Trina | HSV-1 pos. | 11.23 | −1 | −1 | 20.51 | HSV-1 positive |
| 12 | Sample | Trina | HSV-1 pos. | 13.19 | −1 | −1 | 20.25 | HSV-1 positive |
| 13 | Sample | Trina | HSV-1 pos. | 13.10 | −1 | −1 | 20.40 | HSV-1 positive |
| 14 | Sample | Trina | HSV-1 pos. | 15.13 | −1 | −1 | 20.25 | HSV-1 positive |
| 15 | Sample | Trina | HSV-1 pos. | 12.27 | −1 | −1 | 20.35 | HSV-1 positive |
| 16 | Sample | Trina | HSV-1 pos. | 10.90 | −1 | −1 | 20.44 | HSV-1 positive |
| 17 | Sample | Trina | HSV-1 pos. | 12.94 | −1 | −1 | 19.92 | HSV-1 positive |
| 18 | Sample | Trina | HSV-1 pos. | 20.46 | −1 | −1 | 20.51 | HSV-1 positive |
| 19 | Sample | Trina | HSV-1 pos. | 12.51 | −1 | −1 | 20.57 | HSV-1 positive |
| 20 | Sample | Trina | HSV-1 pos. | 15.99 | −1 | −1 | 20.86 | HSV-1 positive |
| 21 | Sample | DLS | HSV-1 pos. | 18.83 | −1 | −1 | 20.82 | HSV-1 positive |
| 22 | Sample | DLS | HSV-1 pos. | 12.29 | −1 | −1 | 20.44 | HSV-1 positive |
| 23 | Sample | DLS | HSV-1 pos. | 9.32 | −1 | −1 | 20.14 | HSV-1 positive |
| 24 | Sample | DLS | HSV-1 pos. | 12.97 | −1 | −1 | 20.19 | HSV-1 positive |
| 25 | Sample | DLS | HSV-1 pos. | 18.07 | −1 | −1 | 20.68 | HSV-1 positive |
| 26 | Sample | DLS | HSV-1 pos. | 11.78 | −1 | −1 | 20.12 | HSV-1 positive |
| 27 | Sample | DLS | HSV-1 pos. | 16.94 | −1 | −1 | 20.63 | HSV-1 positive |
| 28 | Sample | DLS | HSV-1 pos. | 8.50 | −1 | −1 | 20.25 | HSV-1 positive |
| 29 | Sample | DLS | HSV-1 pos. | 16.81 | −1 | −1 | 20.14 | HSV-1 positive |
| 30 | Sample | DLS | HSV-1 pos. | 13.06 | −1 | −1 | 20.99 | HSV-1 positive |
| 31 | Sample | Trina | HSV-2 pos. | −1 | 22.75 | −1 | 21.36 | HSV-2 positive |
| 32 | Sample | Trina | HSV-2 pos. | −1 | 20.26 | −1 | 20.82 | HSV-2 positive |
| 33 | Sample | Trina | HSV-2 pos. | −1 | 16.43 | −1 | 20.74 | HSV-2 positive |
| 34 | Sample | Trina | HSV-2 pos. | −1 | 17.74 | −1 | 20.66 | HSV-2 positive |
| 35 | Sample | Trina | HSV-2 pos. | −1 | 20.57 | −1 | 20.77 | HSV-2 positive |
| 36 | Sample | Trina | HSV-2 pos. | −1 | 12.16 | −1 | 19.94 | HSV-2 positive |
| 37 | Sample | Trina | HSV-2 pos. | −1 | 12.40 | −1 | 20.54 | HSV-2 positive |
| 38 | Sample | Trina | HSV-2 pos. | −1 | 20.26 | −1 | 20.81 | HSV-2 positive |
| 39 | Sample | Trina | HSV-2 pos. | −1 | 14.51 | −1 | 20.12 | HSV-2 positive |
| 40 | Sample | Trina | HSV-2 pos. | −1 | 12.76 | −1 | 20.49 | HSV-2 positive |
| 41 | Sample | Trina | HSV-2 pos. | −1 | 15.69 | −1 | 20.59 | HSV-2 positive |
| 42 | Sample | Trina | HSV-2 pos. | −1 | 16.98 | −1 | 20.59 | HSV-2 positive |
| 43 | Sample | Trina | HSV-2 pos. | −1 | 16.99 | −1 | 20.56 | HSV-2 positive |
| 44 | Sample | Trina | HSV-2 pos. | −1 | 20.38 | −1 | 20.74 | HSV-2 positive |
| 45 | Sample | Trina | HSV-2 pos. | −1 | 11.00 | −1 | 20.77 | HSV-2 positive |
| 46 | Sample | Trina | HSV-2 pos. | −1 | 12.53 | −1 | 21.00 | HSV-2 positive |
| 47 | Sample | Trina | HSV-2 pos. | −1 | 17.39 | −1 | 20.68 | HSV-2 positive |
| 48 | Sample | Trina | HSV-2 pos. | −1 | 13.60 | −1 | 20.35 | HSV-2 positive |
| 49 | Sample | DLS | HSV-2 pos. | −1 | 19.34 | −1 | 20.68 | HSV-2 positive |
| 50 | Sample | DLS | HSV-2 pos. | −1 | 11.13 | −1 | 19.87 | HSV-2 positive |
| 51 | Sample | DLS | HSV-2 pos. | −1 | 13.95 | −1 | 20.25 | HSV-2 positive |
| 52 | Sample | DLS | HSV-2 pos. | −1 | 27.57 | −1 | 20.42 | HSV-2 positive |
| 53 | Sample | DLS | HSV-2 pos. | −1 | 20.22 | −1 | 20.68 | HSV-2 positive |
| 54 | Sample | DLS | HSV-2 pos. | −1 | 26.52 | −1 | 21.00 | HSV-2 positive |
| 55 | Sample | DLS | HSV-2 pos. | −1 | 17.09 | −1 | 20.94 | HSV-2 positive |
| 56 | Sample | DLS | HSV-2 pos. | −1 | 14.62 | −1 | 21.26 | HSV-2 positive |
| 57 | Sample | DLS | HSV-2 pos. | −1 | 8.52 | −1 | 20.91 | HSV-2 positive |
| 58 | Sample | DLS | HSV-2 pos. | −1 | 18.88 | −1 | 20.57 | HSV-2 positive |
| 59 | Sample | DLS | HSV-2 pos. | −1 | 15.75 | −1 | 19.96 | HSV-2 positive |
| 60 | Sample | Trina | VZV pos. | −1 | −1 | 15.10 | 21.34 | VZV positive |
| 61 | Sample | Trina | VZV pos. | −1 | −1 | 17.97 | 21.20 | VZV positive |
| 62 | Sample | Trina | VZV pos. | −1 | 11.12 | 27.33 | 21.00 | VZV and HSV-2 |
| | | | | | | | | positive |
| 63 | Sample | Trina | VZV pos. | −1 | −1 | 11.76 | 21.76 | VZV positive |
| 64 | Sample | Trina | VZV pos. | −1 | −1 | 19.19 | 21.18 | VZV positive |
| 65 | Sample | Trina | VZV pos. | −1 | −1 | 11.55 | 21.29 | VZV positive |
| 66 | Sample | Trina | VZV pos. | 27.77 | −1 | 12.52 | 21.54 | VZV and HSV-1 |
| | | | | | | | | positive |
| 67 | Sample | Trina | VZV pos. | −1 | −1 | 14.04 | 21.26 | VZV positive |
| 68 | Sample | Trina | VZV pos. | −1 | −1 | 17.11 | 21.69 | VZV positive |
| 69 | Sample | Trina | VZV pos. | −1 | −1 | 19.10 | 20.90 | VZV positive |
| 70 | Sample | Trina | VZV pos. | −1 | −1 | 12.93 | 21.15 | VZV positive |
| 71 | Sample | Trina | VZV pos. | −1 | −1 | 13.20 | 21.30 | VZV positive |
| 72 | Sample | Trina | VZV pos. | −1 | −1 | 16.24 | 21.23 | VZV positive |
| 73 | Sample | Trina | VZV pos. | −1 | −1 | 13.03 | 21.39 | VZV positive |
| 74 | Sample | Trina | VZV pos. | −1 | −1 | 18.68 | 20.96 | VZV positive |
| 75 | Sample | Trina | VZV pos. | −1 | −1 | 12.97 | 21.21 | VZV positive |
| 76 | Sample | Trina | VZV pos. | −1 | −1 | 25.92 | 21.03 | VZV positive |
| 77 | Sample | Trina | VZV pos. | −1 | −1 | 22.24 | 20.97 | VZV positive |
| 78 | Sample | Trina | VZV pos. | −1 | −1 | 15.81 | 21.14 | VZV positive |
| 79 | Sample | Trina | VZV pos. | −1 | −1 | 14.02 | 21.10 | VZV positive |
| 80 | Sample | Trina | VZV pos. | −1 | −1 | 15.33 | 21.12 | VZV positive |
| 81 | Sample | Trina | VZV pos. | −1 | −1 | 25.98 | 21.05 | VZV positive |
| 82 | Sample | Trina | VZV pos. | −1 | −1 | 24.09 | 20.79 | VZV positive |
| 83 | Sample | Trina | VZV pos. | −1 | −1 | 14.13 | 21.23 | VZV positive |
| 84 | Sample | Trina | VZV pos. | −1 | −1 | 13.18 | 21.07 | VZV positive |
| 85 | Sample | Trina | VZV pos. | −1 | −1 | 14.85 | 20.93 | VZV positive |
| 86 | Sample | Trina | VZV pos. | −1 | −1 | 15.80 | 20.79 | VZV positive |
| 87 | Sample | Trina | VZV pos. | −1 | −1 | 25.65 | 20.93 | VZV positive |
| 88 | Sample | Trina | VZV pos. | −1 | 33.15 | 13.16 | 21.13 | VZV positive |
| 89 | Sample | Trina | VZV pos. | −1 | −1 | 17.14 | 21.11 | VZV positive |
| 90 | Sample | Trina | VZV pos. | −1 | −1 | 27.58 | 20.85 | VZV positive |
| 91 | Sample | Trina | VZV pos. | −1 | −1 | 16.92 | 20.94 | VZV positive |

### Experimental Results—Fresh Samples

Data from 48 fresh samples are shown in Table 9. The data include results from 22 HSV-1 positive samples that were positive for HSV-1 by both vendor results and byAlinity m® assay results. Results from 26 HSV-2 positive samples are also tabulated that were positive by both vendor results and Alinity m® assay. The samples were tested as “fresh”, and also were tested after 1 freeze/thaw cycle wherein the samples were frozen at −70C or colder temperature, and then thawed at room temperature or at 2-8C prior to testing. There was no impact of a single freeze/thaw on assay results.

TABLE 9
Fresh sample data

| | Test of | Fresh | | After 1 Freeze/Thaw | | Alinity m ® |
|---|---|---|---|---|---|---|
| Sample | Record | HSV-1_CT | HSV-2_CT | HSV-1_CT | HSV-2_CT | Result |
| 1 | HSV-1 POS | 9.11 | −1 | 9.7 | −1 | HSV-1 POS |
| 2 | HSV-1 POS | 9.56 | −1 | 9.24 | −1 | HSV-1 POS |
| 3 | HSV-1 POS | 11.59 | −1 | 13.42 | −1 | HSV-1 POS |
| 4 | HSV-1 POS | 11.66 | −1 | 12.03 | −1 | HSV-1 POS |
| 5 | HSV-1 POS | 11.75 | −1 | 11.42 | −1 | HSV-1 POS |
| 6 | HSV-1 POS | 12.56 | −1 | 12.99 | −1 | HSV-1 POS |
| 7 | HSV-1 POS | 12.59 | −1 | 12.47 | −1 | HSV-1 POS |
| 8 | HSV-1 POS | 12.64 | −1 | 13.03 | −1 | HSV-1 POS |
| 9 | HSV-1 POS | 12.72 | −1 | 12.79 | −1 | HSV-1 POS |
| 10 | HSV-1 POS | 12.87 | −1 | 12.46 | −1 | HSV-1 POS |
| 11 | HSV-1 POS | 13.24 | −1 | 13.22 | −1 | HSV-1 POS |
| 12 | HSV-1 POS | 13.78 | −1 | 13.73 | −1 | HSV-1 POS |
| 13 | HSV-1 POS | 14.43 | −1 | 14.26 | −1 | HSV-1 POS |
| 14 | HSV-1 POS | 14.82 | −1 | 15.11 | −1 | HSV-1 POS |
| 15 | HSV-1 POS | 15.99 | −1 | 15.77 | −1 | HSV-1 POS |
| 16 | HSV-1 POS | 17.56 | −1 | 17.77 | −1 | HSV-1 POS |
| 17 | HSV-1 POS | 17.73 | −1 | 17.49 | −1 | HSV-1 POS |
| 18 | HSV-1 POS | 21.63 | −1 | 21.67 | −1 | HSV-1 POS |
| 19 | HSV-1 POS | 23.63 | −1 | 23.61 | −1 | HSV-1 POS |
| 20 | HSV-1 POS | 28.19 | −1 | 28.41 | −1 | HSV-1 POS |
| 21 | HSV-1 POS | 28.65 | −1 | 28.82 | −1 | HSV-1 POS |
| 22 | HSV-1 POS | 11.04 | −1 | 11.21 | −1 | HSV-1 POS |
| 23 | HSV-2 POS | −1 | 11.36 | −1 | 11.24 | HSV-2 POS |
| 24 | HSV-2 POS | −1 | 12.25 | −1 | 12.6 | HSV-2 POS |
| 25 | HSV-2 POS | −1 | 12.68 | −1 | 13.06 | HSV-2 POS |
| 26 | HSV-2 POS | −1 | 13.72 | −1 | 14.35 | HSV-2 POS |
| 27 | HSV-2 POS | −1 | 14.24 | −1 | 13.92 | HSV-2 POS |
| 28 | HSV-2 POS | −1 | 15.23 | −1 | 15.51 | HSV-2 POS |
| 29 | HSV-2 POS | −1 | 15.99 | −1 | 15.3 | HSV-2 POS |
| 30 | HSV-2 POS | −1 | 16.09 | −1 | 16.13 | HSV-2 POS |
| 31 | HSV-2 POS | −1 | 16.53 | −1 | 16.95 | HSV-2 POS |
| 32 | HSV-2 POS | −1 | 17.15 | −1 | 16.86 | HSV-2 POS |
| 33 | HSV-2 POS | −1 | 17.16 | −1 | 17.21 | HSV-2 POS |
| 34 | HSV-2 POS | −1 | 17.46 | −1 | 17.45 | HSV-2 POS |
| 35 | HSV-2 POS | −1 | 18.02 | −1 | 18.52 | HSV-2 POS |
| 36 | HSV-2 POS | −1 | 21 | −1 | 21.18 | HSV-2 POS |
| 37 | HSV-2 POS | −1 | 21.99 | −1 | 21.9 | HSV-2 POS |
| 38 | HSV-2 POS | −1 | 22.19 | −1 | 21.77 | HSV-2 POS |
| 39 | HSV-2 POS | −1 | 22.24 | −1 | 23.09 | HSV-2 POS |
| 40 | HSV-2 POS | −1 | 22.56 | −1 | 22.6 | HSV-2 POS |
| 41 | HSV-2 POS | −1 | 22.81 | −1 | 22.49 | HSV-2 POS |
| 42 | HSV-2 POS | −1 | 23.15 | −1 | 22.97 | HSV-2 POS |
| 43 | HSV-2 POS | −1 | 27.14 | −1 | 26.4 | HSV-2 POS |
| 44 | HSV-2 POS | −1 | 27.84 | −1 | 27.95 | HSV-2 POS |
| 45 | HSV-2 POS | −1 | 30.64 | −1 | 30.42 | HSV-2 POS |
| 46 | HSV-2 POS | −1 | 31 | −1 | 30.39 | HSV-2 POS |
| 47 | HSV-2 POS | −1 | 31.45 | −1 | 31.29 | HSV-2 POS |
| 48 | HSV-2 POS | −1 | 17.52 | −1 | 17.69 | HSV-2 POS |

These data show that performance with the Alinity m® assay is equivalent for fresh and frozen sample HSV-1, HSV-2 or VZV testing.

We claim:

1. A method of detecting Herpes Simplex Virus 1 (HSV-1), Herpes Simplex Virus 2 (HSV-2) and Varicella Zoster Virus (VZV) nucleic acid in a biological sample from a subject, comprising:
a) extracting nucleic acid from said biological sample;
b) contacting said nucleic acid from said biological sample from said subject with at least two nucleic acid primer pairs selected from the group consisting of SEQ ID NOS: 1 and 2, SEQ ID NOS: 4 and 5, SEQ ID NOS: 7 and 8, SEQ ID NOS: 10 and 11, SEQ ID NOS: 13 and 14, SEQ ID NOS: 16 and 17, and SEQ ID NOS: 19 and 20, and at least two probes selected from the group consisting of SEQ ID NOS: 3, 6, 9, 12, 15, 18 and 21;
d) performing an amplification assay to amplify one or more HSV-1, HSV-2 and VZV nucleic acid targets; and
e) detecting the presence of said HSV-1, HSV-2 and VZV nucleic acid targets in said sample.

2. The method of claim 1, wherein said detecting is binding of at least one said nucleic acid primer or probe to said HSV-1, HSV-2 and VZV nucleic acid.

3. The method of claim 1, wherein said detecting comprises real time polymerase chain reaction (RT-PCR).

4. The method of claim 1, wherein said biological sample is a skin sample, a mucosa sample, an anogenital sample, an oral sample, a cerebrospinal fluid sample and/or a blood sample.

5. The method of claim 1, wherein one or more of said SEQ ID NOS comprises a label.

6. The method of claim 5, wherein said label comprises a fluorophore.

7. The method of claim 5, wherein said label comprises a fluorophore/quencher pair.

8. The method of claim 1, further comprising contacting said biological sample from said subject with a reaction mixture.

9. The method of claim 8, wherein said reaction mixture comprises a reagent selected from a nucleic acid polymerase, a plurality of dNTPs and a buffer.

10. The method of claim 8, wherein said reaction mixture comprises a magnetic microparticle.

11. A composition, comprising at least two nucleic acid primer pairs selected from the group consisting of SEQ ID NOS: 1 and 2, SEQ ID NOS: 4 and 5, SEQ ID NOS: 7 and 8, SEQ ID NOS: 10 and 11, SEQ ID NOS: 13 and 14, SEQ ID NOS: 16 and 17, and SEQ ID NOS: 19 and 20, and at least two probes selected from the group consisting of SEQ ID NOS: 3, 6, 9, 12, 15, 18 and 21.

12. The composition of claim 11, wherein one of more of said SEQ ID NOS comprises a label.

13. The composition of claim 11, wherein said label comprises a fluorophore.

14. The composition of claim 12, wherein said label comprises a fluorophore/quencher pair.

15. The composition of claim 11, wherein said composition is a reaction mixture.

16. A reaction mixture, comprising:
a) at least two nucleic acid primer pairs selected from the group consisting of SEQ ID NOS: 1 and 2, SEQ ID NOS: 4 and 5, SEQ ID NOS: 7 and 8, SEQ ID NOS: 10 and 11, SEQ ID NOS: 13 and 14, SEQ ID NOS: 16 and 17, and SEQ ID NOS: 19 and 20, and at least two probes selected from the group consisting of SEQ ID NOS: 3, 6, 9, 12, 15, 18 and 21; and
b) at least one reagent for performing a nucleic acid amplification reaction.

17. The reaction mixture of claim 16, further comprising a sample.

18. The reaction mixture of claim 17, wherein said sample comprises HSV-1, HSV-2 and/or VZV target nucleic acid.

19. The reaction mixture of claim 16, wherein said reagent is selected from a nucleic acid polymerase, a plurality of dNTPs, and a buffer.

20. The reaction mixture of claim 16, further comprising a magnetic microparticle.

21. The reaction mixture of claim 16, further comprising one or more reagents specific for RT-PCR.

22. A system, comprising:
a) at least two nucleic acid primer pairs selected from the group consisting of SEQ ID NOS: 1 and 2, SEQ ID NOS: 4 and 5, SEQ ID NOS: 7 and 8, SEQ ID NOS: 10 and 11, SEQ ID NOS: 13 and 14, SEQ ID NOS: 16 and 17, and SEQ ID NOS: 19 and 20, and at least two probes selected from the group consisting of SEQ ID NOS: 3, 6, 9, 12, 15, 18 and 21; and
b) an automated sample handling instrument.

23. The system of claim 22, further comprising an assay instrument.

24. The system of claim 23, wherein said assay instrument is a high-throughput assay instrument.

25. The system of claim 22, further comprising reaction components in a multi-container carrier.

26. The system of claim 22, further comprising a microplate thermocycler and/or microplate detector.

27. The system of claim 22, further comprising a microfluidic device.

28. A kit, comprising:
a) at least two nucleic acid primer pairs selected from the group consisting of SEQ ID NOS: 1 and 2, SEQ ID NOS: 4 and 5, SEQ ID NOS: 7 and 8, SEQ ID NOS: 10 and 11, SEQ ID NOS: 13 and 14, SEQ ID NOS: 16 and 17, and SEQ ID NOS: 19 and 20, and at least two probes selected from the group consisting of SEQ ID NOS: 3, 6, 9, 12, 15, 18 and 21; and
b) at least one reagent for performing a nucleic acid amplification reaction.

29. The kit of claim 28, wherein said reagent is selected from a nucleic acid polymerase, a plurality of dNTPs, and a buffer.

30. The kit of claim 28, wherein one or more of said primers or probes comprises a label.

31. The kit claim 30, wherein said label comprises a fluorophore.

32. The kit of claim 31, wherein said label comprises a fluorophore/quencher pair.

33. The kit of claim 28, further comprising a magnetic microparticle.

34. The kit of claim 28, further comprising one or more reagents specific for RT-PCR.

35. The kit of claim 28, further comprising a positive control nucleic acid, a negative control nucleic acid, a calibration nucleic acid, and/or instructions for use of said kit.


SEQUENCE LISTING (21 sequences)

SEQ ID NO: 1 - DNA, 18 bp, synthetic construct
  source 1..18: mol_type = other DNA
accatcgcttggtttcgg

SEQ ID NO: 2 - DNA, 21 bp, synthetic construct
  source 1..21: mol_type = other DNA
ccccagagacttgttgtagga

SEQ ID NO: 3 - DNA, 20 bp, synthetic construct
  source 1..20: mol_type = other DNA
aggcaactgtgctatcccca

SEQ ID NO: 4 - DNA, 18 bp, synthetic construct
  source 1..18: mol_type = other DNA
gaaacagaaacgcgcttg

SEQ ID NO: 5 - DNA, 19 bp, synthetic construct
  source 1..19: mol_type = other DNA
gtccgacgtggcgatgatg

SEQ ID NO: 6 - DNA, 20 bp, synthetic construct
  source 1..20: mol_type = other DNA
tgtagggcgacaggatttgg

SEQ ID NO: 7 - DNA, 17 bp, synthetic construct
  source 1..17: mol_type = other DNA
cggacgatgtttcttgg

SEQ ID NO: 8 - DNA, 23 bp, synthetic construct
  source 1..23: mol_type = other DNA
gcagtgatagcgaagaaatattc

SEQ ID NO: 9 - DNA, 22 bp, synthetic construct
  source 1..22: mol_type = other DNA
tacgaggccccgtccgttattg

SEQ ID NO: 10 - DNA, 19 bp, synthetic construct
  source 1..19: mol_type = other DNA
gatgagggtggttccttcg

SEQ ID NO: 11 - DNA, 18 bp, synthetic construct
  source 1..18: mol_type = other DNA
cccgtctctcgctcgtgg

SEQ ID NO: 12 - DNA, 22 bp, synthetic construct
  source 1..22: mol_type = other DNA
tgtttacggaggcgagttgctg

SEQ ID NO: 13 - DNA, 21 bp, synthetic construct
  source 1..21: mol_type = other DNA
aaaccactaacagcgtctgcc

SEQ ID NO: 14 - DNA, 23 bp, synthetic construct
  source 1..23: mol_type = other DNA
accgaaaatgagggttgttgaac

SEQ ID NO: 15 - DNA, 25 bp, synthetic construct
  source 1..25: mol_type = other DNA
ttgagggaaataaattaccgccgcc

SEQ ID NO: 16 - DNA, 18 bp, synthetic construct
  source 1..18: mol_type = other DNA
cggaggctgctttcacgg

SEQ ID NO: 17 - DNA, 21 bp, synthetic construct
  source 1..21: mol_type = other DNA
ggccgataaacgatgtaacgt

SEQ ID NO: 18 - DNA, 25 bp, synthetic construct
  source 1..25: mol_type = other DNA
tagactggacgatagaggagccagg

SEQ ID NO: 19 - DNA, 28 bp, synthetic construct
  source 1..28: mol_type = other DNA
ctacagcagagttggcagcttcactttc

SEQ ID NO: 20 - DNA, 23 bp, synthetic construct
  source 1..23: mol_type = other DNA
acaaatttggaagccatccatca

SEQ ID NO: 21 - DNA, 24 bp, synthetic construct
  source 1..24: mol_type = other DNA
acgagttcatgagggcaggccgct